(19) United States
(12) Patent Application Publication
(10) Pub. No.: US 2024/0165186 A1
(43) Pub. Date: May 23, 2024

(54) Synergistically Formulated Compound for Mitochondria Supplementation and Enhancement

(71) Applicant: Robert E. Rockwood, Pelham, NH (US)

(72) Inventor: Robert E. Rockwood, Pelham, NH (US)

(21) Appl. No.: 18/506,182

(22) Filed: Nov. 10, 2023

Related U.S. Application Data
(60) Provisional application No. 63/384,244, filed on Nov. 18, 2022.

(51) Int. Cl. A61K 36/424; A61K 9/00; A61K 31/122; A61K 31/4745; A61K 31/706; A61K 31/7084; A61K 35/02

(52) U.S. Cl. CPC A61K 36/424; A61K 9/0014; A61K 9/0053; A61K 31/122; A61K 31/4745; A61K 31/706; A61K 31/7084; A61K 35/02

(57) ABSTRACT

A synergistic composition is provided to support mitochondrial health and ATP production. The composition may be topical and/or ingestible, depending on embodiment. Consistent application of the composition has been shown to produce more healthy, youthful looking skin. The present disclosure results in a unique combination that will nutritionalize and energize skin cells in a way that no other single or multiple compound is capable of doing.

### BACKGROUND OF THE INVENTION

The patent disclosure relates to a skin care formulation for the purpose of improving the appearance of the skin to its smoothest, healthiest and youngest possible attribute.

Skin acts as a natural barrier between internal and external environments and therefore plays an important role in protection against mechanical and chemical injury. The health and appearance of skin deteriorates due to aging, exposure to environmental factors and genetic makeup. The outer skin layer offers the most visible signs of deterioration, however, its deterioration is usually due to the malfunction and reduced function capabilities of the skin cells below the outer skin layer.

The epidermis, the outermost layer of skin, provides a waterproof barrier and creates our skin tone and visible appearance. The epidermis of the skin contains mostly keratin which is constantly exfoliating and renewing itself. The epidermis rests on a supporting dermis that contains the nerve and vascular networks, which nourish the epidermis. The dermis, beneath the epidermis, contains the fibroblast cells which generate collagen and elastin. The dermis also contains connective tissue, hair follicles, and sweat glands. The deeper subcutaneous tissue (hypodermis) is made of fat and connective tissue which directly integrate with other organs within the body. The skin is the largest organ within the body and usually represents up to 15% of the total weight of the body. As long as all of the different cells and functions of all three layers of the skin are operating at top peak, the skin will retain its youthful, healthy and smooth appearance. Skin cells from all three layers are constantly dying and pushing up towards the epidermis where they eventually exfoliate and fall off.

The skin consists of billions of individual cells which are constantly dying off and being replaced by new cells generated via cell division or created by stem cells. The skin replaces itself every 15 days when we are babies, on average every 30 days during our middle years and every 84 days when we are older. As we get older our regenerated cells aren't always as healthy and firm as the cells they replace, and they don't generate as much collagen which is the firming tissue which keeps our skin smooth and younger looking.

With age and deterioration over time, the skin starts decreasing its concentration of cells per inch of surface area along with a decrease in internal cell energy which means less cell regeneration. Less collagen and elastin is produced which decreases the firmness of the skin and allows skin sagging and the formation of wrinkles and reduced skin tone.

### SUMMARY OF THE INVENTION

The subject matter of this application may involve, in some cases, interrelated products, alternative solutions to a particular problem, and/or a plurality of different uses of a single system or article.

In one aspect, an anti-aging skin composition is provided which supports mitochondrial function within the skin cells. The composition comprises active ingredients of pyrroloquinoline quinone (PQQ); ubiquinol CoQ10; shilajit; nicotinamide mononucleotide (NMN) and/or nicotinamide adenine dinucleotide (NAD); and jiaogulan which work synergistically together to provide optimal nutrient support and protection for mitochondrial function. These active ingredients are supported in a carrier for either direct skin application or in an ingestible form such as a pill or powder.

In another specific aspect, this composition may comprise: PQQ in a range of 0.1-0.4 oz per 100 oz. of total composition weight; CoQ10—0.1-0.4 oz per 100 oz. of total composition weight; NMN/NAD—0.01-0.25 oz. per 100 oz. of total composition weight; Shilijit—0.01-0.25 oz. per 100 oz. of total composition weight; and Jiaogulan—1.0-4.0 oz. per 100 oz. of total composition weight.

In yet another aspect, A method for rejuvenating and energizing skin cells is provided, the method involves both topically applying the composition of Claim 1 which has a topical carrier; and orally consuming the composition of claim 1 which has an edible carrier. The topical and edible compositions may both have the same percent ratio of ingredients, with the carrier being different for its different purposes. As such, the composition administration provides nutrients directly to the skin and internally that energize and protect the mitochondria and support active levels of NAD and ATP enzymes in skin cells.

### BRIEF DESCRIPTION OF DRAWINGS

FIG. 1 provides a flowchart of one embodiment of the present disclosure.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

The skin is composed of a number of different types of skin cells that perform different functions. However, all skin cells are energized by the mitochondria, NAD enzymes and the ATP co-enzymes within the cells. This formulation is designed to enhance the energy of the mitochondria, and thus energy of the cells, and maintain higher than typical baseline levels of mitochondrial activity, up to the maximum amount of all of these three enzymes (NAD, ATP, AMPK) in the skin cells. The mitochondria produces the energy which maximizes cell function. ATP acts as the battery or storer of the energy and releases it as necessary per the demands of the cell function. NAD is the enzyme which aids in the efficient transfer of energy.

The term “energizing” mitochondria as well as the term “energizing” cells are both used herein to refer to the ability of the presently disclosed composition to provide protective support to mitochondrial health, provide optimal nutrition to the mitochondria, providing nutrients to revitalize mitochondria as the aging process progresses, and maximizing the effectiveness of the mitochondrial activity. The mechanisms and actions of this “energizing” is discussed further herein.

The subject of this disclosure is a composition which can be administered to a patient, such as a human or other mammal that will nutritionalize the mitochondria and NAD enzymes, as well as the ATP co-enzymes which are the powerhouse energy producers for skin cells. When all three of these enzymes are healthy and active, they energize, protect and increase the number of healthy skin cells which in turn aids in the production of collagen and elastin. Collagen and Elastin are a key component in giving our skin a healthy youthful appearance. Unfortunately, as we age the number of skin cells in our body get reduced and their mitochondria may decrease, causing energy levels to slow down. Revitalizing the skin cells combined with inducing new growth is the most powerful way to reduce the appearance of aging, wrinkling skin while alternately increasing the youthful look and glow of the skin. The present invention has identified five ingredients which are synergistic with each other as to stimulating the skin cells. When they are combined in a single compound for application/consumption by a patient, each ingredient enhances and improves the effectiveness of the other. Synergistic compounding is to mix together two or more substances to produce a combined effect greater than the sum of their separate effects. The present composition is the result of that engineering premise and results in a unique combination that will nutritionalize and energize skin cells in a way that no other single or multiple compound is capable of doing.

The five synergistic ingredients are:
1) Pyrroloquinoline Quinone (PQQ)
2) Ubiquinol CoQ10 (Ubidecarenone, also called coenzyme Q10, is a 1,4-benzoquinone)
3) Shilajit
4) NMN are the initials for Nicotinamide Mononucleotide, and/or NAD which are the initials for nicotinamide adenine dinucleotide
5) Jiaogulan

This patent application relates to the combination of the above ingredients whether they are used in a topical skin care cream or a topical serum or a topical oil as well as their usage in an oral nutrition skin pill or other oral administration for internal digestion.

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

The purpose of this unique composition is to provide a powerful compound that will aid in energizing skin cells to rejuvenate, protect and uplift the mitochondrial and NAD elements within the skin cells. As we age, we not only experience a reduction in the number of our skin cells, but we also have a significant reduction in the strength of the energy within each cell. This is what contributes to the aging look of our skin. The skin thins out and causes a sagging and wrinkling effect. Strong and healthy cells provide strong and healthy looking skin. A portion of our cells are defined as fibroblast cells. The fibroblast cells are the major producer of collagen. Collagen is a skin protein. As the number of fibroblast cells are reduced due to aging, our healthy collagen content is reduced significantly. Collagen is to the skin as a foundation is to a house. The stronger the foundation below makes everything on top of it more stable, smooth, and secure. Reducing the collagen is the same as reducing the foundation.

In addition, as we grow older the skin sees the same thing happening to its elastin content. Elastin is the protein that gives the skin its bounce back properties. Without elastin the skin loses its ability to maintain, retain and regain a smooth look. When both your collagen and elastin levels are maintained at high levels, your skin will maintain a healthy and youthful appearance due to a strong smooth texture.

The best way, in fact the only effective way to maintain a healthy and youthful skin appearance, is to energize the skin cells by energizing the mitochondria and increasing NAD content within these cells. This present disclosure helps achieve that goal to its maximum by providing the nutrients that energize the mitochondria and support active levels of NAD.

As stated above, this composition was specifically formulated with five ingredients that not only work together, but more importantly act in a synergistic manner when combined in a single compound. Synergy occurs when two or more substances interact to produce a combined effect greater than the sum of their separate effects. Research has determined that combining these five ingredients together substantially increases the positive effect of each ingredient. The combination of all five in a single compound could increase the sum total effectiveness of the ingredients by themselves as much 50 times or more. In other words, the five individual ingredients of 1 plus 1 plus 1 plus 1 plus 1 does not add up to 5, but instead synergizes to an effective level of 50.

The five ingredients utilized in this disclosure for synergistic effect are the following:

Pyrroloquinoline Quinone (PQQ) acid base. PQQ is a multi-functional nutrient that is commonly found in plant foods. It was first discovered in 1964 as a cofactor for enzymatic reactions. In simple terms a cofactor helps enzymes accomplish their job. The enzyme job that PQQ aids is critical to making the skin look its best and appear its youngest. This job specifically is to enhance the energy and production of mitochondria.

Mitochondria is found inside all of the body's cells, including the skin cells. It is the one element needed to produce our primary source of cell energy, also known as adenosine triphosphate (ATP). ATP provides 95% of the cellular energy that powers all living functions in our body. Unfortunately, a byproduct of this energy generation is the formation of huge amounts of free oxygen radicals which are extremely destructive to the “good” molecules in our body. We are all familiar with rusting of steel structures. The rusting of steel is caused by the same chemical reaction that ages or “rusts” the human body. Free oxygen radicals break down steel, free oxygen radicals break down human cells and causes disease and aging of the body.

Free radicals attack the structure of our cell membranes, creating metabolic waste products that disturb DNA and RNA production, interfere with the synthesis of protein, and destroy important cellular enzymes.

The mitochondria are easy targets because they are located exactly where these free radicals are produced. In the case of skin cells, mitochondria destruction is directly related to the aging process as our cells degenerate.

As with many functions in our body, when we are young our body can fight off free radicals and they don't do as much damage. As we get older our protective functions slow down and cell destruction increases which we see as the aging process. The rate of cellular production of two free radicals—superoxide anions and hydrogen peroxide—significantly increases, attacking mitochondria the most.

Scientists started discovering back in the mid 1960's that PQQ supplements could help in the slowing down and even reversing the free oxidant radical attacks. Many people take vitamin C supplements as an anti-oxidant which certainly provides some aid. It should be noted that whole food derived Vitamin C is effective in truly fighting free radical oxidation. Most skin care products and oral nutritional supplements used a chemical version of Vitamin C that is even less effective in fighting oxidation. PQQ has been shown to be as much as 1000 times more effective than vitamin C as an antioxidant.

Mitochondria is located on the edge of the cell which makes it prone being attacked and destroyed by free oxygen radicals which are always invading our body. PQQ is one of the most powerful, if not the most powerful antioxidant nutrient in the world. Therefore, the PQQ reduces the amount of free radicals floating in the body and therefore reduces mitochondria destruction, thereby supporting and protecting mitochondria in skin (and other) cells. This allows the mitochondria to proliferate because of a slowed destruction rate. More mitochondria means less aging and healthier looking skin.

Ubiquinol CoQ10 (Ubidecarenone, also called coenzyme Q10, is a 1,4-benzoquinone). It is an essential cofactor in mitochondrial oxidative phosphorylation which is the final step necessary to produce ATP (i.e. “cell energy”). When we are young our body produces lots of CoQ10. As we grow older, we produce less and less of it. Coq10 is another anti-oxidant and works even more efficiently when combined with PQQ. However, CoQ10 also has another very important function, it increases energy transfer capabilities. In other words, it not only helps protect the mitochondria, it also helps in making the mitochondria work faster and more efficiently. CoQ10 occurs in two forms—Ubiquinol and Ubiquinone. It has been discovered that the more effective form is Ubiquinol. There are many supplements of CoQ10 in the Ubiquinone form which requires the body to transform it into Ubiquinol. Aging makes this harder and harder for the body to do. This invention uses the Ubiquinol form of Coq10 so that the body doesn't have to work to transform it into its most effective form.

Combining CoQ10 with PQQ provides the beginning of a positive synergistic combination to aid the production and energizing of the Mitochondria. PQQ protects the mitochondria from damage, supporting more mitochondria in the cells, and CoQ10 provides an essential cofactor to the protected and supported mitochondria to spur additional ATP production per mitochondrion. This creates a synergy of more mitochondria than baseline without supplementation, each generating higher ATP compared to baseline without supplementation.

Shilajit. This substance is found primarily on rocks in the Himalayas. It is formed over centuries by the decomposition of plants. As previously mentioned CoQ10 energizes and protects the mitochondria. However, it does it by depleting itself. CoQ10 neutralizes the flood of free radicals by donating one of its own electrons to the heathy cells and thereby reducing the number of free cells generated during cellular energy production. This process of electron donation eventually weakens the capability of the CoQ10.

By adding Shilajit to the composition, it actually preserves and revitalizes the Ubiquinol CoQ10, replenishing lost electrons so the CoQ10 maintains high activity and sees a much slower depletion effect. Shilijit aids in the transfer of electrons from various proteins in the body to the CoQ10, and therefore makes these electrons available to be infused into the depleted CoQ10. Adding Shilajit increases the synergistic effect of the total compound by allowing the CoQ10 to be more effective and provide longer lasting effects.

NMN are the initials for Nicotinamide Mononucleotide. This is a nutritional supplement that triggers an increase in the biosynthesis of the coenzyme NAD+ (nicotinamide adenine dinucleotide). By middle age, our NAD+ levels have plummeted to half that of our youth. Numerous studies have demonstrated that boosting NAD+ levels can reverse mitochondrial dysfunction. In other embodiments, direct NAD supplementation may be used in addition to or instead of the NMN.

A recent report published under the auspices of the World health Organization Across stated that an increase in intracellular levels of NAD+ triggers shifts that enhance human survival, including boosting cellular energy levels (i.e. ATP as noted below) and upregulating cellular repair. The aging process is sped up when NAD levels are low and slowed down when NAD levels are increased. NAD+ supplementation via NMN or NAD even further supports mitochondrial health and provides proper nutrient support for ATP generation, again providing a synergy with the above noted elements by resulting in more, more effective mitochondria.

Jiaogulan. This herb aids in activating an enzyme called AMPK and making it more bio-available. AMPK becomes activated as soon as it senses a decrease in ATP (the energy enzyme in your skin cell). The AMPK then promotes a catabolic pathway to generate more ATP. Thus, higher levels of ATP result in an increase in ATP levels. The more ATP (energy) in your cells, the more mitochondria your body can maintain and produce. It also deactivates many destructive effects of aging to keep your cells youthful and vital. It's been used in Asia for over 600 years with exceptionally positive results.

As made clear from the above definitions, each of the ingredients not only increases the capability of the other, but just as importantly it maximizes the full intent of the formulation to make sure that the mitochondria, which is the skin cell power generator, is energized, protected from dying off in large numbers and enables the skin cells to reach their highest levels of capability within the body. The combination of elements results in more, more healthy mitochondria as well as supporting above-baseline levels of ATP production by each mitochondrion.

The PQQ provides the antioxidant function to keep the mitochondria alive.

The CoQ10 also is an antioxidant but becomes a more powerful antioxidant when combined with PQQ. CoQ10 has another function which is to support and enable better energy transfer capabilities to the mitochondria so it enhances the cell function to be more efficient. Unfortunately, as the CoQ10 performs this function, much like a working battery, it tends to deplete itself and becomes weaker over time.

The Shilajet acts on the CoQ10 like a battery charger acts on a battery. It keeps the CoQ10 at peak levels and minimizes the natural depletion in the CoQ10.

The NMN increases the NAD levels in the body. With higher NAD levels there is less mitochondrial dysfunction which allows the CoQ10 to more efficiently donate electrons into both the mitochondria and the skin cell itself rather than to the already-dysfunctional mitochondria. Again, direct supplementation with NAD is also contemplated by this invention and may be used as a replacement for or supplementation to the NMN.

The Jiaogulan helps to increase an enzyme called ATP by activating AMPK. The more ATP you have in your body the more mitochondria can be hosted in each skin cell which in turn provides the CoQ10 a larger storage base to provide maximum energy to each skin cell for a healthier skin capability. It also enhances the antioxidant capabilities of both CoQ10 and the PQQ.

In order to maximize the synergistic impact of all of the ingredients required the development of a specialized manufacturing process to ensure no detrimental processing methods were used that could destroy the natural nutritional value of each ingredient and its ability to intermingle with the other ingredients.

PQQ and NMN/NAD are a powder formation. By grinding and rolling the powder it reduces to a smooth non particle composition. These powders could be directly mixed with any vegan butter such as Shea or Mango. However it is preferable to mix it in a Vegetable Glycerin base which allows the powder to reach an almost liquified form. The glycerin serves as a thick liquid base which can also absorb the liquid form of CoQ10 and Shilajit. Jiaogulan is a leafy plant which requires a minimum of a 30 day maceration process by immersing it into virgin organic olive oil which is the best medium for absorbing all of the nutrients out of the leaves. The macerated Jiaogulan can then be added to the vegetable glycerin base which contains the other four ingredients. All of the ingredients must be kept at room temperature or below during the entire manufacturing process to ensure that none of them are altered or suffer nutrients being cooked out which occurs beginning at 118 degrees F. Synergizing is maximized by using low speed mixing blades for a minimum of 30 minutes.

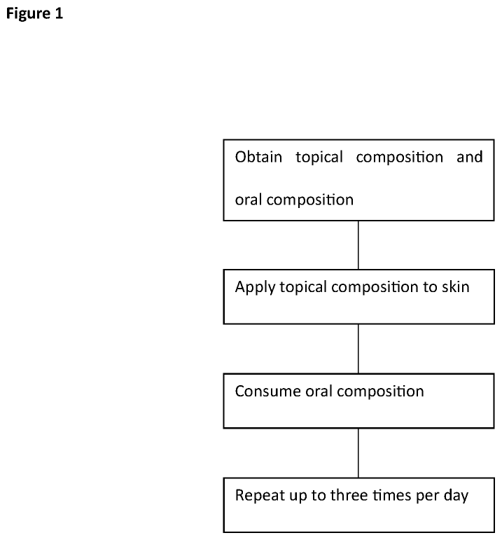

If we look at a cross section of human skin cell as shown in FIG. 1, we can see how the Mitochondria is usually position on the outer edges of the cell itself.

This positioning puts it in the least protected area of the cell. It makes it prone to being easily damaged especially when harmful chemicals and oxidants penetrate the skin cells. When oxidants first enter a cell, they are at full strength, much like a hurricane when it first makes landfall. Just like the houses on the shoreline usually experience the maximum damage, the mitochondria are exposed to maximum damage. As we age, and more oxidants attack the skin cells over time, the more the reduction of the number of mitochondria in our skin cells. Combining this with two other aging factors, namely as the body ages and beginning as early as age 21, the human body produces less new or replacement mitochondria along with a reduction of energy levels within each mitochondrion. Mitochondria are the powerhouse of all living cells. Reduce the number of mitochondria along with the energy contained within each enzyme and you will see the appearance of more aging on the skin.

The same oxidation destruction and aging effectiveness reduction occurs with the NAD and ATP enzymes.

The formulation defined in this patent application is designed to add nutritional supplements, as well as vitamin, bioflavonoid and enzyme supplements to the mitochondria, NAD and ATP enzymes specifically to aid cells in re-energizing and reproducing themselves with the overall purpose of giving a youthful and healthy appearance to the skin. The formulation also provides supplemental nutrients to protect and fight off harmful oxidation.

This compound can be formulated in varying ranges of volume combinations for each of the five ingredients. The range of mixture volume can be anywhere from 5% to 50% of each individual ingredient.

Although the synergistic formulation would greatly aid the mitochondria function if it was just applied topically, its positive effects could be increased even further if it was combined with an oral application. The oral application could be in the form of either a liquid or a capsule containing the same synergistic formulation. At the present time there are no skin care products that offer the same formulation for both topical and simultaneous oral application.

The outer layer of skin, the epidermis, serves as a protective barrier to prevent penetration into the skin and body of outside elements. This penetration protection applies to the simplest of elements like water up to and including the most toxic bacteria. However, it is widely understood and recognized that if the element has a micron size particle of 80 nm (nanometers) or smaller it will easily penetrate the epidermis. If a skin care topical cream contains highly active ingredients. even if the particle size is larger than 80 nm it can still penetrate the epidermis and dermis layers of the skin. If the topical application is made in a hot or warm environment the pores of the skin opens wider and therefore allows a topical cream or serum to penetrate deeper and faster. The same is true if the topical is applied after a hot shower or bath or steam room event. The skin pores open up and will allow a topical application of any kind to penetrate deeper into the skin.

The synergistic formulation described in this application contains both active and sufficiently small particles which will penetrate into the skin regardless of the skin temperature. It will also penetrate deeper when the application is made while the skin is at higher temperatures. However, it is possible that not all of the ingredients will reach down into the hypodermis, or bottom third layer of the skin for maximum penetration. The formulation is still significantly effective when just lying on the epidermis or first layer of the skin. Its effectiveness increases as it enters the dermis or second layer of skin. The dermis contains the cells that produce collagen and elastin. Collagen gives skin its solid foundation for a smooth look and reduction of wrinkles. Elastin gives the skin it's flexibility and rebounding capability for a total bounce back even after it is squeezed by contact or compressed by clothing articles. The advantage of a topical application over an oral application is that the individual can directly target those areas of the skin that have maximum damage or potential future damage. Obviously that includes thin skin areas such as around the eyes, lips and neck. It also includes all those areas that are directly exposed to the environmental elements like the sun and the atmosphere. Those areas would include the face. neck and hands. An oral application cannot be targeted to a specific area of the body but basically spreads to the body at large.

Some of the topical agents may reach the hypodermis or third layer of the skin but not in full capacity. The hypodermis is the fat layer of the skin which stores collagen. It also contains connective tissue and blood flow which is a direct path to all of the body organs. The hypodermis is a support layer for the two outermost skin layers—the dermis and epidermis. It provides additional aid in keeping the outer layers looking healthy and smooth. Topical elements can provide some nutritional value to the hypodermis but not a maximum amount.

In order to maximize the nutritional capabilities of the synergistic formulation on the hypodermis it is best to apply the formula in a manner of combinations.

One combination is to apply it topically both with a cream and a serum. A serum is already in liquid form when applied and therefore by definition will have a more penetrating capability than a solid cream. Applying a topical serum before applying a topical cream with the same formulation will give the synergistic formulation a deeper penetration into the skin. In such embodiment, the “active” ingredients noted herein are mixed with a carrier. The carrier may have a “base” such as glycerin or equivalent, and also has additional inactive components such as those known in the art for topical skincare compositions. Similarly, an edible composition also has the active ingredients mixed into a carrier base, and may have additional components such as those known in the art for edible compositions.

Another combination is to apply the described synergistic formulation via a topical cream and/or a topical serum in combination with an oral application of the same synergistic formulation. The oral application bypasses the protective epidermis skin barrier and reaches the hypodermis layer directly by feeding into the blood flow system. Using an oral and topical combination of the synergistic formulation improves its effectiveness and anti-oxidant protection to all three layers of the skin. This can be referred to as an I/E (Internal/External application). An I/E will maximize the rejuvenation of all the skin cells from top to bottom.

The % volume range of the five components

| | |
|---|---|
| PQQ | 5-50% |
| CoQ10 | 5-50% |
| Shilajit | 5-50% |
| NMN/NAD | 5-50% |
| Jiaogulan | 5-50% |

In various embodiments, it may be preferable to have the PQQ and CoQ10 at higher levels if the focused intention of the formulation is to primarily energize and protect the mitochondria of cells. In other embodiments, if the intention is to transfer the energy generated by the mitochondria (ATP) as quickly as possible than it is best to have the NMN/NAD, Shilajit and Jiaogulan at slightly higher levels.

The chart above shows % ranges of the five components relating to the % volume of the total volume of the components, rather than % range of the components as part of the carrier as a whole, which may vary substantially. In a cream or serum form, the components will be a much smaller fraction, compared to, for example an edible form, which will have less, or even no carrier. In one topically applied or edible embodiment, the five ingredients may comprise approximately 1-10 oz. of the combined compounds per 100 oz. of total composition, with the balance being a carrier (cream, serum, and the like for topical embodiments, or ingestible carrier for edible embodiments). In another topically applied or consumable embodiment, the five ingredients may comprise approximately 2-4 oz of the combined compounds per 100 oz. of the composition, with the balance being a carrier. The five components may be in an equal ratio in one embodiment.

In edible/orally consumed embodiments, the combined amount of the five components may range from 1-10 oz. per 100 oz. of total composition. In another embodiment, the combined five components may range from 2-4 oz. per 100 oz. of total composition. The ratio of components may be similar to that discussed above with respect to the topical compositions.

In one embodiment, the present composition may relate to the combination of all five ingredients regardless of their individual percentage of the total volume within the compound. In a further embodiment, the composition may be a formulation in various media, such as the formulation in a topical cream, topical serum, or an ingestible pill or powder. Depending on embodiment, topical vs ingestible compositions may have higher or lower percentages of the total composition, though in other embodiments, the topical and ingestible compositions may have the same percentage of the total composition. In one embodiment, it was unexpectedly found that the combined ingredients making up the same composition of both topical and ingestible embodiment yielded optimal results. In this embodiment, the combined ingredients may make up 1-6% of the total composition, with the remainder of the weight being a carrier, which may vary depending on embodiment and use. The present disclosure also contemplates ranges that are within the noted ranges and explicitly also discloses these ranges, such as 2-6%, 1-3% and so on.

In one specific exemplary embodiment, the composition may comprise the following composition ranges per 100 oz of composition (balance being a carrier as described above): PQQ—0.1-0.4 oz; CoQ10—0.1-0.4 oz; NMN—0.01-0.25 oz; Shilijit—0.01-0.25 oz; Jiaogulan—1.0-4.0 oz. This provides for a total of 1.22-5.29 oz. per 100 oz of topical or edible composition.

It has been unexpectedly discovered that the above-noted five ingredients, when combined and administered together, enhance each other to provide a synergistic increase in cellular energy and anti-oxidant levels of supplementation that cannot be provided by any of the ingredients if applied by themselves.

Generally, the present disclosure relates to and covers the synergistic formula composition described above, and its use for the purpose of providing a supplement geared towards the energizing and protecting the mitochondria, NAD and ATP enzymes to aid in improving the appearance of the skin.

In varying embodiments, the five ingredients noted above may be combined in any carrier media including, but not limited to, vegan oils and/or natural butters, synthetic oils and/or synthetic butters and/or synthetic pastes, gels, glycerin and/or powder bases. In one embodiment, the above-noted synergistic composition may be formulated into a solid cream base. In another embodiment, the composition may be formulated as a serum liquid base. In some further embodiment, both a solid cream base embodiment and a serum liquid base embodiment may both be applied topically to the skin at the same day.

In some embodiments, it has been discovered that quality results of use may be achieved using only three or four of the ingredients combined together. Certain combinations still were found to provide a synergistic supplementation for the mitochondria and may be combined in any configuration without straying from the scope of this invention. For example using the three components of PQQ, CoQ10 and Shilijit or PQQ, CoQ10 and NMN would still have a synergistic positive effect on the skin for the reasons discussed above. Combination of all five even further improve synergistic effects.

In one embodiment of use, the above-noted synergistic composition may be administered as an oral medium, i.e. an edible composition, which can include, but is not restricted to pills, powders. liquids or gels and that the oral composition would be taken alone or in combination with the topical application during the same day or other time period.

Preparation of certain embodiments may be performed as follows—the ingredients are combined into a singular carrier base, such as glycerin and mechanically mixed together. Once mixed, the active ingredients and glycerin base are then mixed with the remainder of the carrying agents (which vary depending on topical or edible embodiments). Mixing the five active ingredients with the glycerin ensures that the active ingredients are closely integrated, maximizing synergistic potential, before getting diluted into the rest of the mixture.

In some embodiments, the composition may use the same active formulation in both an internal (consumed) and external (topical) medium for application to the body.

Turning now to FIG. 1, a flow chart of one embodiment of administration of the synergistic composition is provided. Initially, both a topical composition such as those discussed herein, as well as an orally consumable composition such as those discussed herein is obtained. A user may then apply the topical composition to skin, and consume the oral composition, providing both external and internal support to the skin cells. This process may be repeated periodically such as weekly, bi-weekly, once daily, twice daily, three times daily, and the like.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A composition for enhancing the energy and functionality of skin cells:
pyrroloquinoline quinone (PQQ);
ubiquinol CoQ10;
shilajit;
nicotinamide mononucleotide (NMN) and/or nicotinamide adenine dinucleotide (NAD);
jiaogulan; and
a carrier.

2. The composition of claim 1 wherein the carrier is a solid cream base or a serum liquid base.

3. The composition of claim 1 wherein the carrier is an edible carrier.

4. The composition of claim 1 wherein the composition is formed as an edible oral medium, wherein the carrier is a material suitable for human consumption, and wherein the composition comprising at least one of a pill, powder, liquid, gel.

5. The composition of claim 1 wherein the carrier comprises glycerin.

6. The composition of claim 1 wherein the combination of ingredients comprises equal ratios of PQQ, ubiquinol CoQ10, shilajit, NMN and/or NAD, and jiaogulan.

7. The composition of claim 1 wherein the PQQ, ubiquinol CoQ10, shilajit, NMN and/or NAD, and jiaogulan together comprise between 1-6% of a weight of the composition, with the balance the carrier.

8. The composition of claim 1 wherein the PQQ, ubiquinol CoQ10, shilajit, NMN and/or NAD, and jiaogulan together comprise between 2-4% of a weight of the composition, with the balance the carrier.

9. The composition of claim 1 wherein the composition comprises:
PQQ in a range of 0.1-0.4 oz per 100 oz. of total composition weight;
CoQ10—0.1-0.4 oz per 100 oz. of total composition weight;
NMN/NAD—0.01-0.25 oz. per 100 oz. of total composition weight;
Shilijit—0.01-0.25 oz. per 100 oz. of total composition weight;
Jiaogulan—1.0-4.0 oz. per 100 oz. of total composition weight.

10. A method of preparation of a composition for rejuvenating and energizing skin cells comprising the steps of:
combining PQQ, ubiquinol CoQ10, shilajit, NMN and/or NAD, and jiaogulan;
mechanically mixing the combined components with a carrier base; and
combining the mixed components and carrier base with a remainder of components of a carrier to form the composition for rejuvenating and energizing skin cells.

11. The method of claim 10 wherein the carrier base is glycerin.

12. The method of claim 10 wherein the combined components comprise:
PQQ in a range of 0.1-0.4 oz per 100 oz. of total composition weight;
CoQ10—0.1-0.4 oz per 100 oz. of total composition weight;
NMN/NAD—0.01-0.25 oz. per 100 oz. of total composition weight;
Shilijit—0.01-0.25 oz. per 100 oz. of total composition weight;
Jiaogulan—1.0-4.0 oz. per 100 oz. of total composition weight.

13. The method of claim 10 wherein the PQQ, ubiquinol CoQ10, shilajit, NMN and/or NAD, and jiaogulan together comprise between 1-6% of a weight of the total composition, with the balance the carrier base and the remainder of components of the carrier.

14. The method of claim 10 wherein the PQQ, ubiquinol CoQ10, shilajit, NMN and/or NAD, and jiaogulan together comprise between 2-4% of a weight of the total composition, with the balance the carrier base and the remainder of components of the carrier.

15. A method for rejuvenating and energizing skin cells, comprising:
topically applying a topical composition comprising: pyrroloquinoline quinone (PQQ);
ubiquinol CoQ10; shilajit; nicotinamide mononucleotide (NMN) and/or nicotinamide adenine dinucleotide (NAD); jiaogulan; and a topical carrier; and
orally consuming an edible composition comprising: pyrroloquinoline quinone (PQQ); ubiquinol CoQ10; shilajit; nicotinamide mononucleotide (NMN) and/or nicotinamide adenine dinucleotide (NAD); jiaogulan; and an edible carrier;
wherein the composition provides nutrients directly to the skin and internally that energize and protect the mitochondria and support active levels of NAD and ATP enzymes in skin cells.

16. The method of claim 15 wherein the both the topical composition and the edible composition components comprise:
PQQ in a range of 0.1-0.4 oz per 100 oz. of total composition weight;
CoQ10—0.1-0.4 oz per 100 oz. of total composition weight;
NMN/NAD—0.01-0.25 oz. per 100 oz. of total composition weight;
Shilijit—0.01-0.25 oz. per 100 oz. of total composition weight;
Jiaogulan—1.0-4.0 oz. per 100 oz. of total composition weight.

17. The method of claim 15 wherein the PQQ, ubiquinol CoQ10, shilajit, NMN and/or NAD, and jiaogulan of both the topical composition and edible composition each comprise between 1-6% of a weight of the total composition, with the balance the carrier base and the remainder of components of the carrier.

18. The method of claim 15 wherein the PQQ, ubiquinol CoQ10, shilajit, NMN and/or NAD, and jiaogulan of both the topical composition and edible composition each comprise between 2-4% of a weight of the total composition, with the balance the carrier base and the remainder of components of the carrier.

19. The method of claim 15 wherein the topical carrier is a solid cream base or a serum liquid base.

20. The method of claim 15 wherein the edible carrier comprising at least one of a pill, powder, liquid, gel.
